(19) United States
(12) Patent Application Publication
(10) Pub. No.: US 2007/0197962 A1
(43) Pub. Date: Aug. 23, 2007

(54) CATHETER FOR REMOVING FOREIGN SUBSTANCE IN BLOOD VESSEL

(76) Inventor: Masuo Morikawa, Nara (JP)

Correspondence Address: HESLIN ROTHENBERG FARLEY & MESITI PC, 5 COLUMBIA CIRCLE, ALBANY, NY, 12203, US

(21) Appl. No.: 11/677,135

(22) Filed: Feb. 21, 2007

(30) Foreign Application Priority Data
Feb. 22, 2006 (JP) 2006-046052

(51) Int. Cl. A61M 31/00; A61M 29/00; A61M 37/00

(52) U.S. Cl. 604/96.01; 604/93.01

(57) ABSTRACT

A catheter for removing foreign substances in a blood vessel includes a catheter main body and an expansion part that is provided spaced from a front end of the catheter main body at a predetermined front end distance and expands a contour of the catheter, and is particularly suitable to be inserted from downstream of the bloodstream. The side face of the catheter main body has a side hole for suction within the area of the front end distance. It is advisable that the catheter has a plurality of holes which are provided equally spaced centering an extension axis of the catheter main body (at symmetrical positions on a cross section vertical to the extension axis). The catheter for removing foreign substances in a blood vessel capable of preventing outflow of foreign substances downward from the part to be treated is provided.

### FIELD OF INVENTION

The present invention relates to a catheter for removing foreign substances such as thrombi (including plaques) in a peripheral blood vessel.

### DESCRIPTION OF PRIOR ART

As a peripheral blood vessel protection device (a protection device) in an endovascular treatment (an intervention) of a peripheral blood vessel, a multi-vessel device for conducting a catheter which is provided with a bloodstream blocking balloon is known, for example, as disclosed in Japanese Unexamined Patent Publication No. 2005-312654. As shown in FIG. 15, this includes a main conduit and a bloodstream blocking balloon attached on the outer circumference of the main conduit near its free end, and the main conduit has independent first and second conduction holes therein. The bloodstream blocking balloon is designed to be expanded or contracted by a pressure of a fluid to be supplied through the second conduction hole, and a thrombus or the like in the blood vessel is got rid of by suction through the first conduction hole together with blood.

### SUMMARY OF THE INVENTION

However, at a front end of the main conduit of the foregoing conventional multi-vessel device, in order to reliably block the bloodstream by the bloodstream blocking balloon, the bloodstream blocking balloon is provided at a position rear (downstream side) from the front end of the main conduit to some extent. In treatment such as removal of thrombi and the like, a suction catheter is inserted through with a main conduit aperture on the front end of the main conduit ahead. Therefore, between the suction catheter for sucking thrombi and the bloodstream blocking balloon exists an insuctionable area where the suction cannot be carried out (see FIG. 15). This causes the problem such that thrombi and plaques not entering the main conduit but flowing off to the insuctionable area neighboring the main conduit accumulate on a blocking surface which is formed by blocking of the bloodstream blocking balloon.

When the accumulated thrombi and plaques in the insuctionable area flow toward the end of a peripheral blood vessel due to contraction of the bloodstream blocking balloon, a patient comes to be in a very critical condition. In other words, even a rather fine plaque is liable to block bloodstream because the blood vessel is subdivided and heighten the possibility of occurrence of a bloodstream disorder. In addition to this, since a size of an insertion tube and a place where the tube can be inserted are limited, it is very difficult to treat a peripheral thin blood vessel. Therefore, downstream outflow of thrombi and/or plaques from the place treaded or to be treated due to the intervention puts a patient in a very critical condition and such condition is a serious medical problem.

It is therefore an object of the present invention to provide a catheter for removing foreign substances in a blood vessel capable of preventing thrombi from flowing out downstream from a place treaded or to be treated.

In order to solve the foregoing problem, the following means of (1) to (4) are adopted.

(1) The catheter for removing foreign substances in a blood vessel of the present invention includes a catheter main body, an expansion part that is provided spaced from a front end of the catheter main body at a predetermined front end distance and expands a contour of the catheter, and a side hole for suction of foreign substance in a blood vessel that is provided on a side face of the catheter main body within an area of the front end distance.

(2) In the foregoing catheter, the side hole has a plurality of holes which are provided equally spaced centering an extension axis of the catheter main body (at symmetrical positions on a cross section vertical to the extension axis).

(3) Further, the catheter for removing foreign substance in a blood vessel of any of the foregoing catheters may have the expansion part which includes a flow path for fluid or gas that is annularly disposed along an outer circumference of the catheter main body, and an inflating and deflating bag that communicates with a front part of the flow path and is annularly disposed along the outer circumference of the catheter main body, and expandable and contracible in response to inflow and outflow of the liquid or the gas passing through the flow path.

(4) In any of the catheters of (1) and (2) stated above, the expansion part may include an umbrella body with a spread sheet that is disposed on the outer circumference of the catheter main body and a binder for holding the umbrella body in a binding state in which the umbrella body is being bound along the side face of the outer circumference of the catheter main body, and the spread sheet spreads to expand or tense further outwardly from the side face of the outer circumference of the catheter main body so as to block a bloodstream in a release condition where the umbrella body is not bound by the binder and relaxes to loosen or contract into a binding state where the umbrella body is bound by the binder.

With the structures stated above, the catheter for removing a foreign substance or substances in a blood vessel according to the present invention can suck foreign substances (including plaques and thrombi) in a blood vessel held back and accumulated from the side hole, and prevent or stop foreign substances in a blood vessel from flowing into the downstream blood vessel from a place to be treated.

With the structures stated above, the catheter for removing a foreign substance or substances in a blood vessel according to the present invention can prevent the outflow of foreign substances in a blood vessel to the downstream blood vessel from a place to be treated.

### BRIEF DESCRIPTION OF THE DRAWINGS

FIG. 1 is a perspective explanatory diagram of a catheter for removing foreign substances in a blood vessel according to Embodiment 1 of the present invention.

FIG. 2 is a side explanatory diagram in partial and axial cross section of the catheter for removing foreign substances in a blood vessel according to Embodiment 1 of the present invention.

FIG. 3 is a front view in section taken along the line A-A of the catheter for removing foreign substances in a blood vessel shown in FIG. 2.

FIG. 4 is a front view in section taken along the line B-B of the catheter for removing foreign substances in a blood vessel shown in FIG. 2.

FIG. 5 is a explanatory side diagram in axial section showing the catheter for removing foreign substances in a blood vessel in use according to Embodiment 1 of the present invention.

FIG. 6 is a side view in section of the catheter for removing foreign substances in a blood vessel according to Embodiment 2 of the present invention.

FIG. 7 is an explanatory side diagram in axial section of the catheter for removing foreign substances in a blood vessel according to Embodiment 3 of the present invention.

FIG. 8 is an explanatory front diagram in axial section of the catheter for removing foreign substances in a blood vessel according to Embodiment 4 of the present invention.

FIG. 9 is an explanatory side diagram partially and axially in section showing a binding state of the catheter for removing foreign substances in a blood vessel according to Embodiment 5 of the present invention.

FIG. 10 is an explanatory side view in axial section showing the catheter for removing foreign substances in a blood vessel in use according to Embodiment 5 of the present invention.

FIG. 11 is an explanatory side view partially in section showing a release state of the catheter for removing foreign substances in a blood vessel according to Embodiment 5 of the present invention.

FIG. 12 is an explanatory side view in axial section of the catheter for removing foreign substances in a blood vessel according to Embodiment 5 of the present invention.

FIG. 13 is an explanatory side view partially in section showing a release state of the catheter for removing foreign substances in a blood vessel according to Embodiment 6 of the present invention.

FIG. 14 is an explanatory side view partially in section showing a release state of the catheter for removing foreign substances in a blood vessel according to Embodiment 7 of the present invention.

FIG. 15 is an explanatory side view in axial section of an example of a main conduit of a conventional catheter-conducting multi-vessel device.

### BEST MODE FOR CARRYING OUT THE INVENTION

Hereinafter, the preferable embodiments of the present invention will be described with reference to the drawings showing respective embodiments.

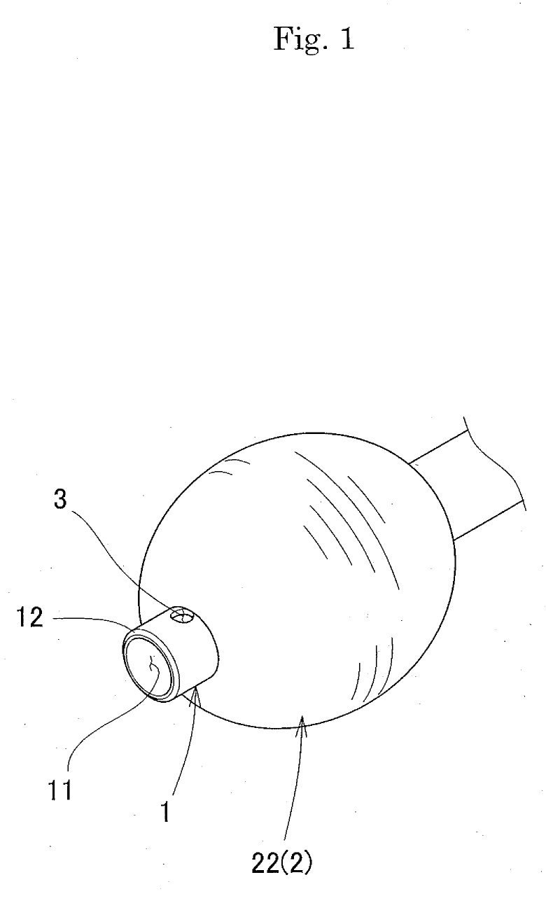

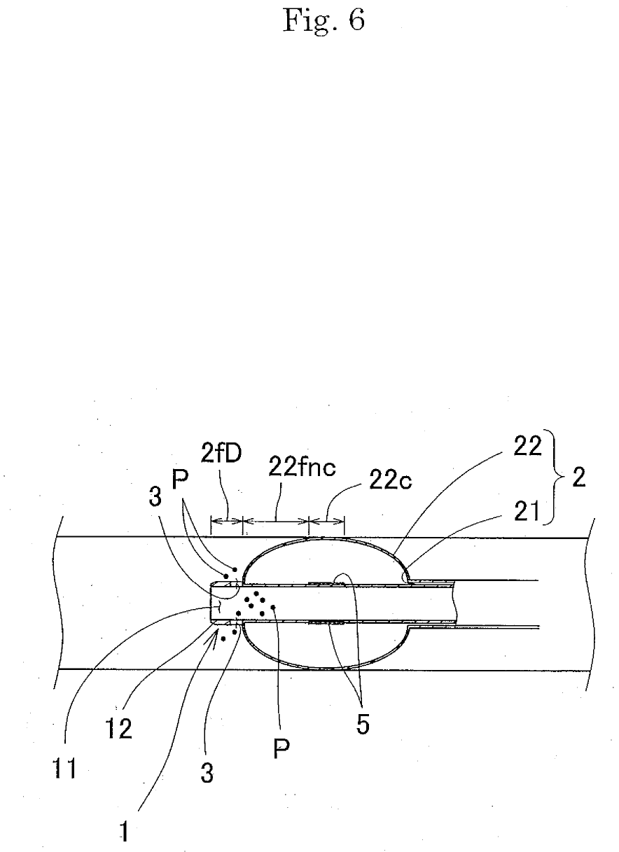

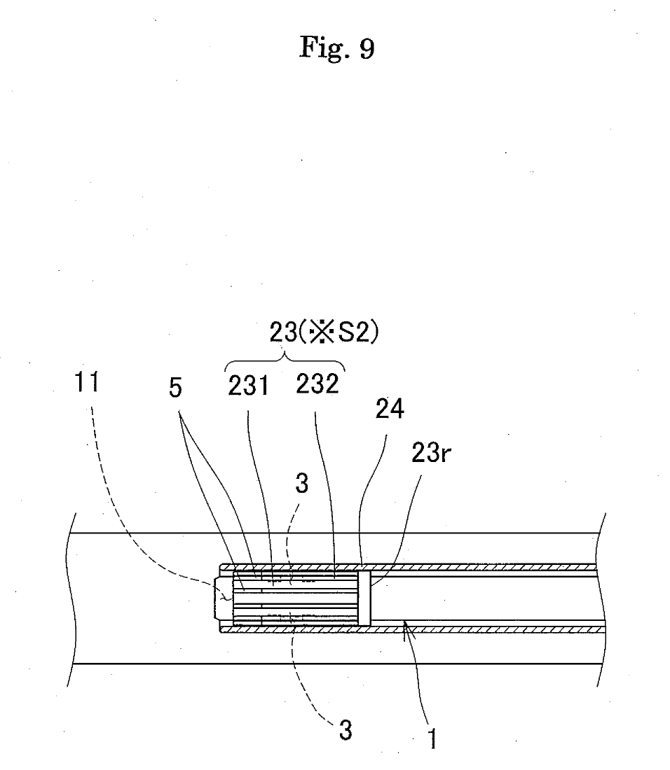

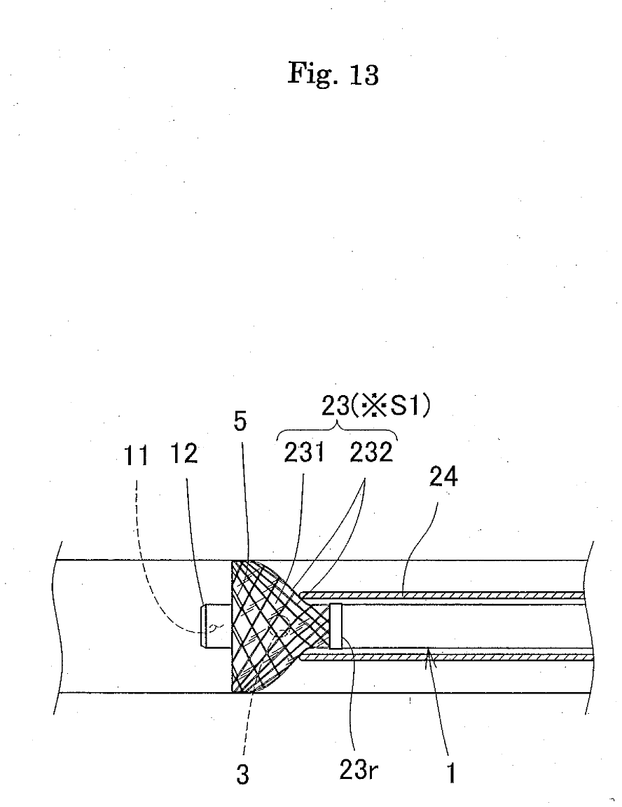

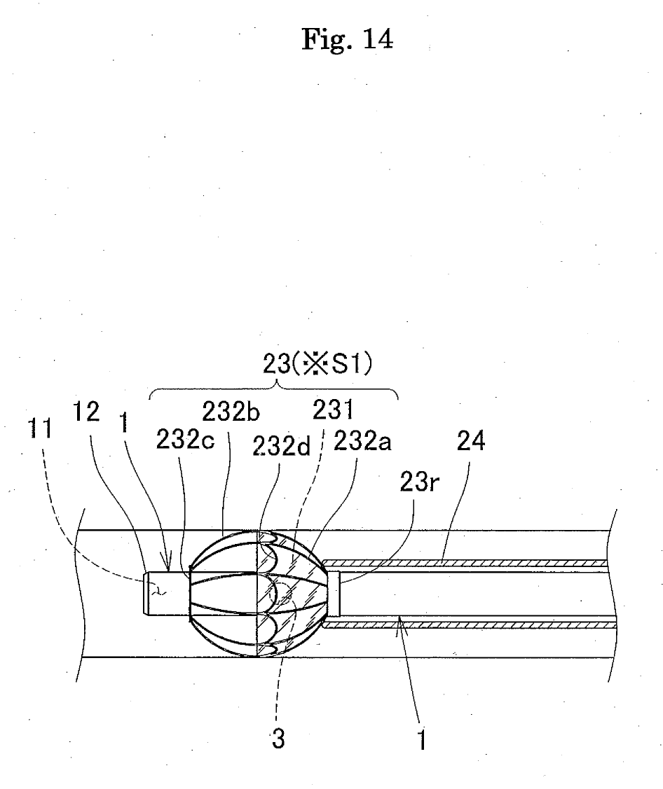

FIGS. 1 to 5 show a catheter for removing foreign substances in a blood vessel according to Embodiment 1 of the present invention. FIGS. 6 to 8 respectively show catheters for removing foreign substances in a blood vessel according to Embodiments 2 to 5 of the present invention. FIGS. 9 to 12 show a catheter for removing a foreign substance in a blood vessel according to Embodiment 5 of the present invention, and FIGS. 13 and 14 respectively show catheters for removing foreign substances in a blood vessel according to Embodiments 6 and 7 of the present invention.

In any embodiment, the catheter for removing foreign substances in a blood vessel according to the present invention includes a catheter main body 1 and an expansion part 2 which is spaced from the front end of the catheter main body 1 at a predetermined front end distance 2fD and expands the outer contour of the catheter. And the catheter main body 1 has a side face on which a side hole 3 for suction (a side suction hole 3) is formed in the predetermined area of the front end distance 2fD.

Further, in the catheters for removing foreign substances in a blood vessel according to Embodiments 4 and 5 (FIGS. 1 to 8), the expansion part 2 which expands the outer contour of the catheter includes a flow path 21 that is annularly disposed along the outer circumference of the catheter main body 1, through which a liquid or a gas can flow; and an inflating and deflating bag 22 that is annularly provided along the outer circumference of the catheter main body 1 and inflates and deflates in response to the inflow and outflow of the liquid or the gas passing through the flow path 21.

On the other hand, in the catheters for removing foreign substances in a blood vessel according to Embodiments 5 to 7 (FIGS. 9 to 14), the expansion part 2 which expands the outer contour of the catheter includes an umbrella body 23 that is disposed on the outer circumference of the catheter main body and has a spread sheet 231; and a binder 24 that holds the umbrella body 23 in a binding state S2 where the umbrella body 23 is bound so as to be along the outer circumferential face of the catheter main body 1.

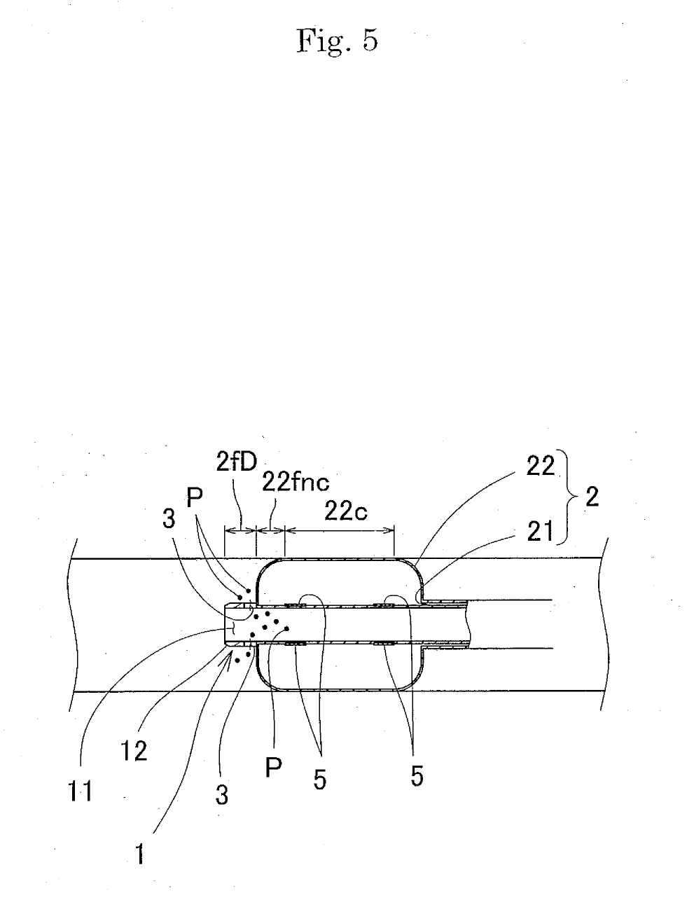

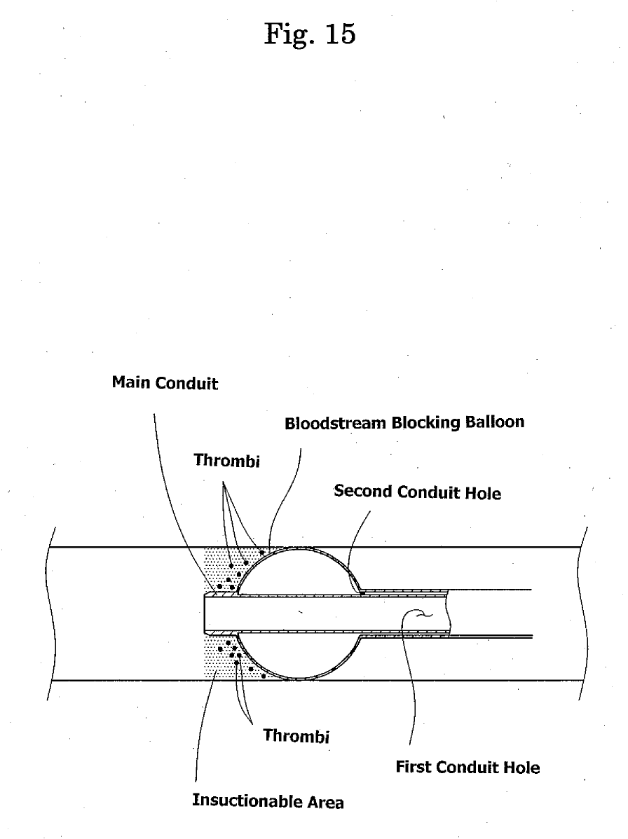

FIG. 15 shows an example of a main conduit of a conventional catheter-conducting multi-vessel device. Near the free end of the main conduit in FIG. 15, foreign substances in a blood vessel P remain accumulated more in an insuctionable area on the side face of the front end, compared with FIGS. 5 to 7 or FIG. 12.

The catheter for removing foreign substances in a blood vessel according to the present invention is a catheter for removing foreign substances in a blood vessel, such as thrombi (including plaques) in a peripheral blood vessel, by suction, and the catheter itself has a suction function on its front end face and the its side face. The catheter for removing foreign substances in a blood vessel is a concept including catheters referred to as a percutaneous catheter for removing thrombi and a catheter for discharging clots. Since having the suction function on its front end face and its side face, the present catheter is particularly suitable to be used being introduced from the downstream blood flow, however, also can be used inserting from the upstream blood flow.

The present invention is different from an introduce catheter in which the catheter itself also has a sheath introducer function, however, by inserting a dilator in a lumen 11 at the front end portion of the catheter main body 1, the present invention can be also used as a catheter for removing foreign substances in a blood vessel having an introduce function.

### Embodiment 1

The catheter main body 1 is made of a catheter pipe which is extended remaining in an annular shape, has the lumen 11 along an extension axis and its front end is open by the circular lumen 11. The periphery of the front end face is provided with a chamfered portion 12 which is obliquely chamfered in an extending direction and with respect to an axial vertical surface. In other words, on the chambered portion 12, the outer shape of the catheter main body 1 is tapered toward the front end in the axial direction.

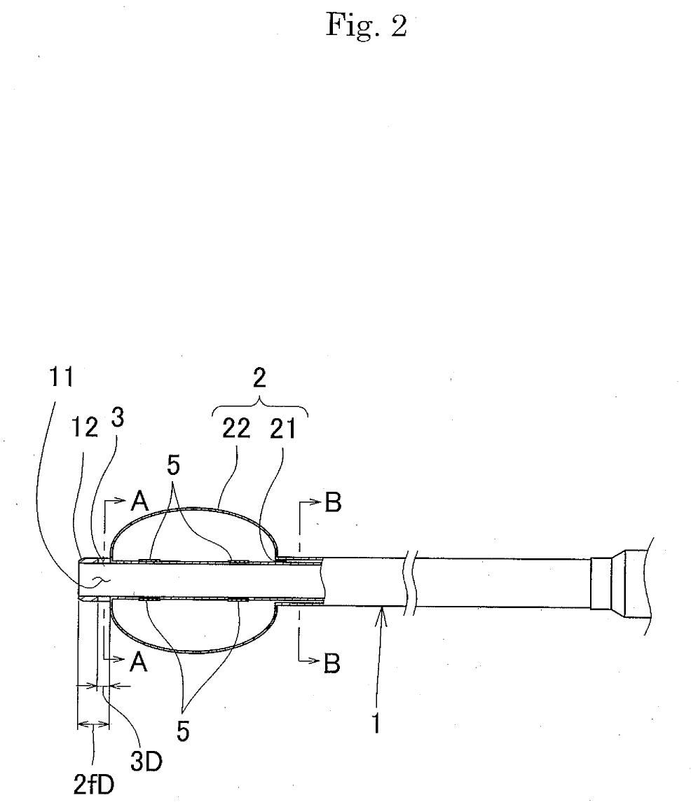

As shown in FIGS. 2 and 5, the expansion part 2 is spaced from the front end of the catheter main body 1 at a predetermined front end distance 2fD so as to expand the outer contour of the catheter. It is preferable that the predetermined front end distance 2fD is at least a half of the diameter or more of the lumen 11 of the catheter main body 1, or, for example, the diameter or more of the lumen 11.

Specifically, the inflating and deflating bag 22 defining the front end side of the expansion part 2 in Embodiment 1 is provided on a rear position spaced from the front end of the main body 1 at the predetermined front end distance 2fD. Thereby, when the catheter is being inserted, the inflating and deflating bag 22 in a contracted state is prevented from bursting and tearing when touching a skin or the like, and a large resistance due to the inflating and deflating bag 22 is eliminated so as to provide smooth insertion of the catheter.

The expansion part 2 of Embodiment 1 has a flow path 21 that is provided annularly around and along the circumference of the catheter main body 1 and fluid or gas passes through the flow path; and the inflating and deflating bag 22 that is provided annularly along and around the circumference of the catheter main body 1, communicates with the front of the flow path 21, and is expandable and contractible in response to the inflow and outflow of the fluid or the gas. The liquid or the gas causes to expand or contract the inflating and deflating bag by flowing out from or flowing into the inflation and deflation bag.

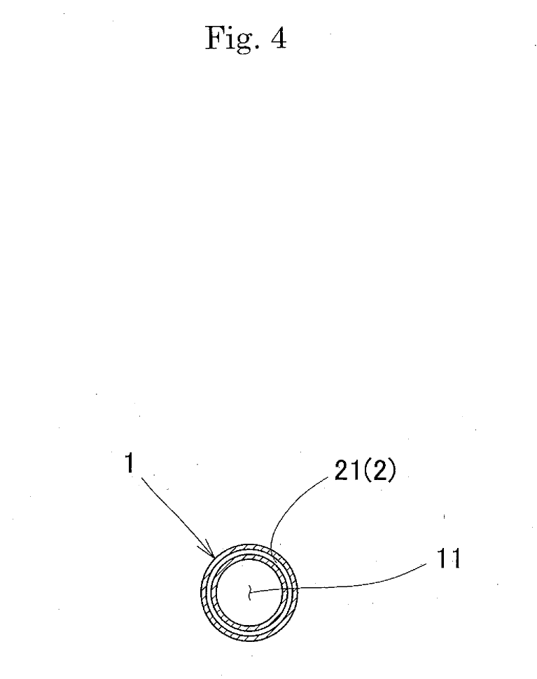

The flow path 21, as seen from the side view in section including an axis, is annularly disposed around and along the extending direction of the catheter main body 1, and fluid or gas can flow through and within the flow path. According to the embodiment, the flow path 21, as seen from the front view in section vertical to the axis shown in FIG. 4, the flow path 21 forms a tubular and thin annular fine gap which is coaxial with the axial of the lumen 11, and is disposed so as to be around the circumference of the lumen 11, i.e. on the outer circumference of the catheter main body 1. In other words, the flow path 21 forms double circles in the front view in section and a so-called coaxial lumen structure is formed with the lumen 11 of the catheter main body 1 and this flow path 21.

The inflating and deflating bag 22 communicates and connects with the front of the flow path 21 and is disposed annularly around the circumference of the catheter main body 1. The bag 22 itself can expand and contract in response to the outflow and inflow of fluid or gas from the flow path 21. FIGS. 1 to 3 and 5 only show a expanded state. Under the condition that little or no liquid or gas is present, the inflating and deflating 22 stands in the contracted state. Here the bag surface forming the inflating and deflating bag 22 relaxes and loosens to contact with and lie on the outer circumferential face of the catheter main body (not illustrated).

In addition, as shown in FIG. 1, in the expanded state, the inflating and deflating bag 22 forms an oval sphere having an expanding axial direction of the catheter main body 1 as its longitudinal axis. However, in practical use, as shown in FIG. 5, in a side view in section including the axis, a blood vessel contacting portion 22c of the inflating and deflating bag comes to contact with a blood vessel and remains so without forcing the vessel to expand more than as it is. The contacting portion 22c, not less than a half of the entire longitudinal length of the inflating and deflating bag 22 (preferably, for example, about ¾ thereof), is formed in the longitudinal center of the bag. The bag flexibly corresponds to the configuration of the interior surface of a blood vessel, by which more reliable blocking of the blood vessel is achieved.

Further, here again referring to FIG. 5, a portion from the front end of the vessel contacting portion 22c up to that of the inflating and deflating bag 22 is defined as a no-contacting front portion 22fnc, then this no-contacting front portion 22fnc is formed with a curvature, for example, larger than a circular arc having a center angle 60 degrees. In addition, in order to prevent accumulation of foreign substances in a blood vessel, it is advisable that the portion of the axial center side of the no-contacting front portion 22fnc, in an expanded state, forms an approximately vertical surface, for example, within ±15 degrees with respect to the extension axis. Further, it is preferable that this portion is approximately vertical with the outer circumferential face of the catheter main body.

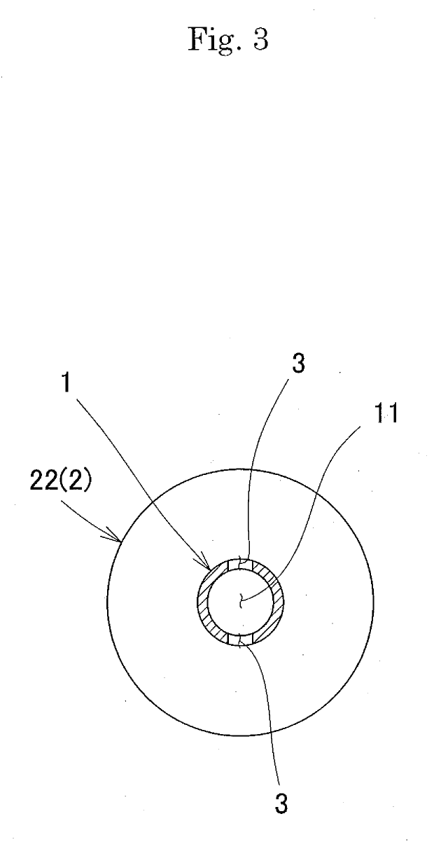

Centering the extension axis of the catheter main body 1, a plurality of side holes 3 are provided equally spaced at symmetrical positions in a vertical cross section with respect to the extension axis. In Embodiment 1, as shown in FIG. 3, two side holes 3 are provided at the symmetrical positions that are on the same position in terms of the axis direction, and the hole diameter 3D is, for example, about ⅓ of the diameter of the lumen 11 of the catheter main body 1.

In addition, as shown in FIGS. 2 and 5, it is preferable that the side holes 3 are disposed spaced from the front end of the catheter main body 1 at a predetermined axial distance (namely, the length left after subtracting the hole diameter 3D from the front end distance 2fD of the inflating and deflating bag 22) and not abut on the front end of the catheter main body 1. With this structure, the front end portion of the catheter main body 1 on which particularly a large outer force is applied keeps a strength no less than predetermined, and is prevented from being neither curved nor deformed during the insertion of the catheter main body 1. In the embodiment, the no-contacting front portion 22fnc of the inflating and deflating bag 22 abuts with the base end edge of the side hole 3, and foreign substances P in a blood vessel P can be more reliably collected.

As shown in FIG. 2, the catheter main body 1 or the expansion part 2 is provided with a marker 5 showing a front side and a rear side in an axial direction of the expansion part 2. This marker 5 is made of metal such as platinum and tantalum which can be viewed under sight through of an X ray, and is formed so as to wind around the catheter main body 1. As shown in FIG. 5, the front end and the rear end of the marker 5 respectively correspond to the front end and the rear end of the blood vessel contacting portion 22c, and the marker 5 has a front part and a rear part at two positions which respectively have viewable predetermined axial directional lengths.

### Embodiment 2

FIG. 6 is a side view in section of the catheter for removing foreign substances in a blood vessel (in an expanded state) in use according to Embodiment 2 of the present invention. In the catheter for removing foreign substances in a blood vessel of Embodiment 2, as compared with Embodiment 1, the expanding diameter of the inflating and deflating bag 22 is smaller and the length of the blood vessel contacting portion 22c is about ⅕ of the axial (longitudinal) length of the inflating and deflating bag 22. In addition, the marker 5 is made of only one part, positioned on the center in the axial (longitudinal) length of the inflating and deflating bag 22 and corresponding to the front and rear ends of the shorter blood vessel contacting portion 22c. In such a structure, the marker 5 can reliably indicate an axial center of the blood vessel blocking position. The no-contacting front portion 22fnc of the inflating and deflating bag has an axial length longer than that of Embodiment 1, and, as angles of the curvature with respect to the axis become smaller as the bag expands from the center part of the axis toward the outer circumferential side. In the vicinity where the bag contacts with a blood vessel, the non-contacting front portion 22fnc forms a fine angle with respect to the interior surface of the blood vessel so as to prevent foreign substances P from accumulating there. Other main structures are equal to those of Embodiment 1.

### Embodiment 3

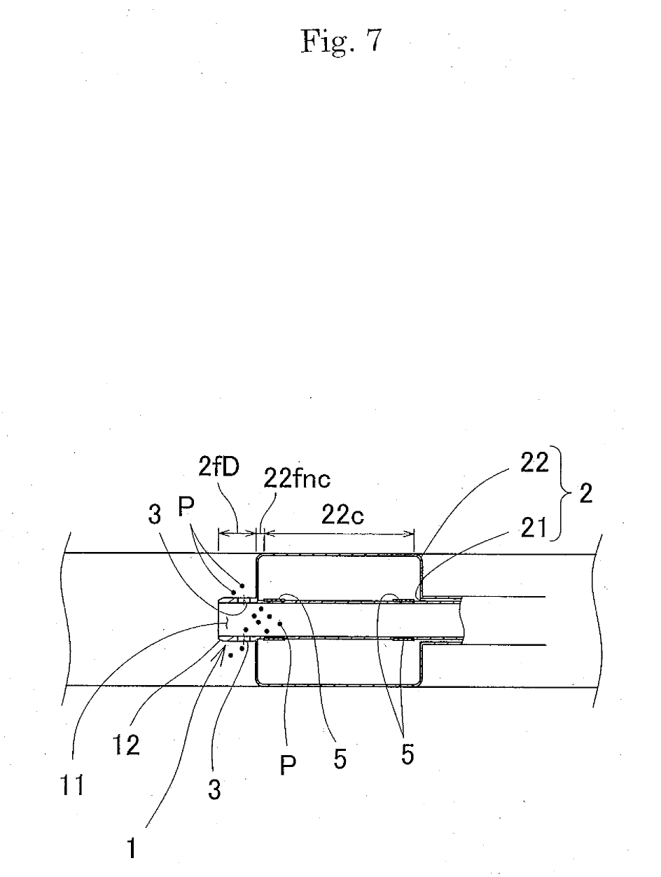

FIG. 7 is a side view in section of the catheter for removing foreign substances in a blood vessel (in a expanded state) in use according to Embodiment 3 of the present invention. In the catheter for removing foreign substances in a blood vessel of Embodiment 3, as compared with Embodiment 1, the inflating and deflating bag 22 in the expanded state forms approximately a circular cylinder, not an oval sphere. However, as shown in FIG. 7, the front and rear ends of the blood vessel contacting portion 22c form curved boundaries in an arc so as to prevent the inflating and deflating bag 22 from bursting, breaking, and tearing due to deviation of the force from outside. In this embodiment, the blood vessel contacting portion 22c extends almost the full length of the entire area (specifically, for example, not less than 80%) in the axial (longitudinal) length of the inflating and deflating bag 22 and this enables a more reliable blocking of a blood vessel. In addition, the no-contacting front portion 22fnc has a small curved face and a substantially plane face perpendicular to the axis from the axial center up to near the outer circumference, and in this structure foreign substances P in a blood vessel are less prone to accumulate there. Other main structures are equal to those of Embodiment 1.

### Embodiment 4

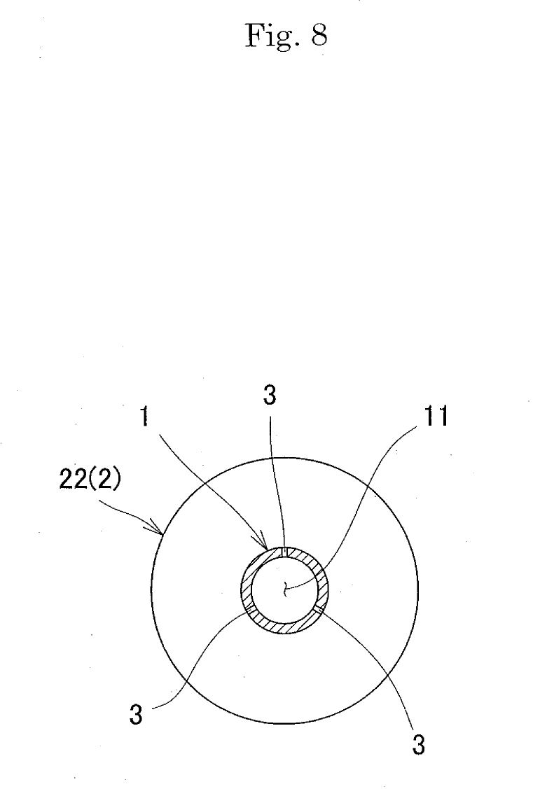

FIG. 8 is an explanatory diagram of a front view in axial section of the catheter for removing foreign substances in a blood vessel according to Embodiment 4 of the present invention. This is a sectional view vertical to the axis at the position having the side holes and corresponding to the A-A position of FIG. 2 showing Embodiment 1. Three side holes 3 in Embodiment 4 are provided at the same intervals in the same axial position. A hole diameter 3D is approximately ⅛ of the diameter of the lumen 11 of the catheter main body 1 so that the catheter has a predetermined strength neither to be readily deformed nor crushed. Other main structures are equal to those of Embodiment 1.

### Embodiment 5

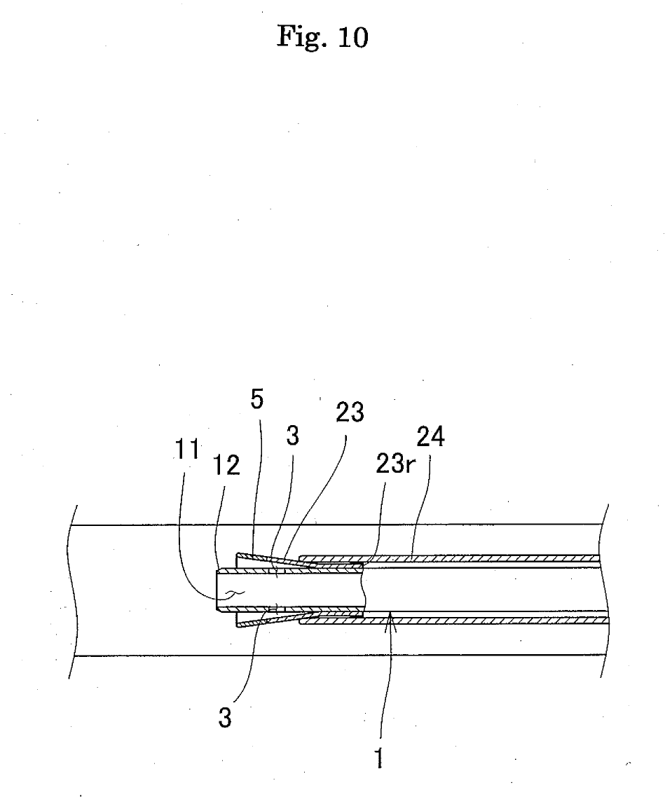

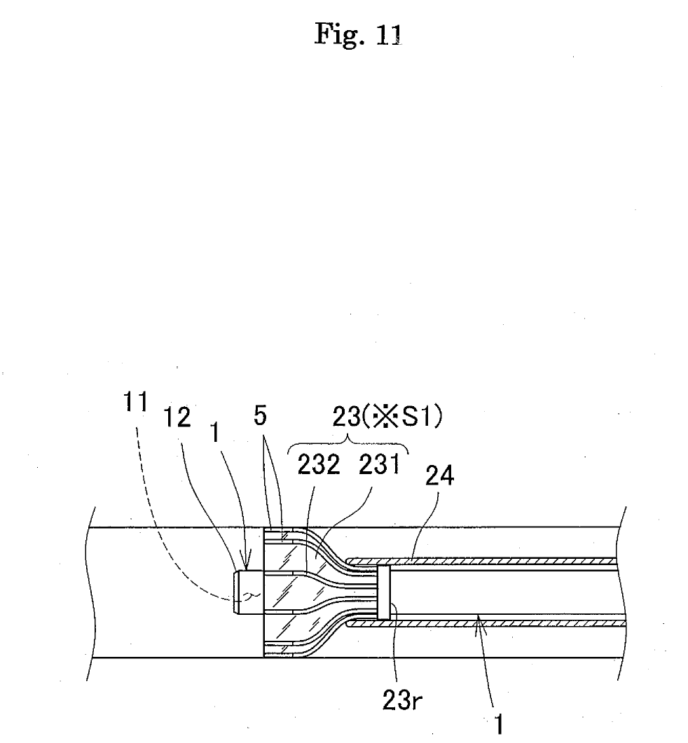

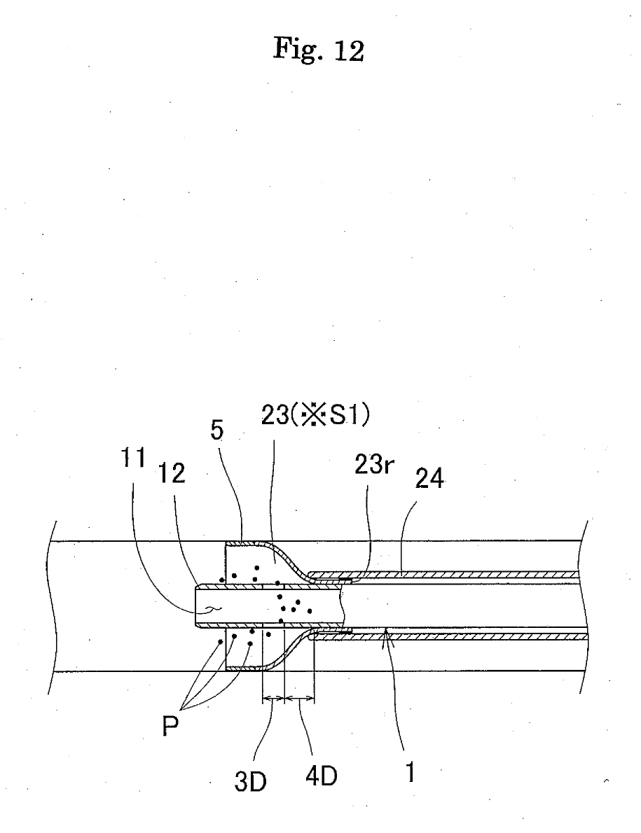

FIG. 9 is an explanatory diagram showing a side view partly and axially in section of the catheter for removing foreign substances in a blood vessel in a binding state S2 according to Embodiment 5 of the present invention. In FIG. 9, only a blood vessel and a binder 24 are shown in cross section. FIG. 10 shows a state in use subsequent to FIG. 9 as an explanatory diagram of a side view in axial section. FIG. 10 shows a state in which the binder 24 is sliding axially toward the base end side following the state shown in FIG. 9 and an umbrella body 23 is expanding. Then, FIG. 11 shows a state in use subsequent to FIG. 10 in an explanatory diagram of a side view partly in section. In FIG. 11, the binder 24 has farther slid axially toward the base end side following the state in FIG. 10 and a peripheral end of the umbrella body 23 and the portion of a spread sheet 231 near the peripheral come in contact with a blood vessel. Here, the umbrella body 23 stands in a release state S1. FIG. 12 is an explanatory diagram of a side view in axial section of the catheter for removing foreign substances in the blood vessel in the release state S1 of FIG. 11.

The expansion part 2 of the catheter for removing foreign substances in a blood vessel of Embodiment 5 has the umbrella body 23 that is provided with the spread sheet 231 and disposed on the outer circumference of the catheter main body 1; and the binder 24 that holds and keeps the umbrella body 23 in the binding state S2 where the body 23 is bound so as to be on and along the outer circumferential face of the catheter main body 1.

In the release state S1 where the umbrella body 23 is not bound by the binder 24, the spread sheet 231 of the umbrella body 23 opens outwardly further from the outer circumferential face of the catheter main body 1 so as to block the bloodstream and tenses or expands (FIGS. 11 and 12). On the other hand, the umbrella body being bound by the binder, the spread sheet 231 becomes to relax and loose up to the binding state S2 (FIG. 9). Further, the spread sheet 231 may be expandable as it tenses while being opened, contractible as it relaxes or loosens while being closed, or contractible without being loose.

Specifically, the umbrella body 23 is provided with spread sheet 231 and spread ribs 232. The spread sheet 231 has an open end in the side of the front end of the catheter and a fixed end in the side of the base end thereof, and opens like an umbrella. The spread ribs, which are fixed to the spread sheet 231, keep the spread sheet 231 open in a release state S2. Both of the spread sheet 231 and the spread ribs 232 are secured on a fixing portion so as to abut with the outer circumference of the catheter main body 1. This spread sheet has minute pores which allow blood components to pass through but not allow thrombi or plaques to pass through.

The spread ribs 232 can be made of shape-memory alloy, which memorizes the shape thereof when the umbrella body 23 is spread in the release state S1. The spread ribs 232 of Embodiment 5 are made of a plurality of shape-memory alloy pieces extending in parallel along the axis so as to be along the axis in the binding state S2. In the release state S1, the spread ribs 232 extend and expand from the fixed end abutting with the outer circumferential surface of the catheter main body 1 toward the front end in the axial direction and outwardly so that the distance from the axis becomes large.

The binder 24 has a tubular body, which covers the fixing portion of the umbrella body 23 and surrounds the umbrella body 23 and the catheter main body 1, and is disposed axially slidable in the rear of the umbrella body at the side of the base and in the position where it can abut with the umbrella body 23. Sliding the binder 24 keeping the front end of the binder 24 abutting with the umbrella body 23 allows to adjust a degree of expansion due to the release of the umbrella body 23. Here, it is preferable that a base end 23r of the fixing portion of the umbrella body 23 is kept within of the binder 24 even in the release state S1 of the umbrella body 23 shown in FIGS. 11 and 12.

As shown in FIG. 12, it is preferable that the side holes 3 of Embodiment 5 are disposed forward from a reference position or a contact position between the umbrella body 23 and the catheter main body 1, and spaced from the reference position at a predetermined hole edge base end distance 4D. In other words, this hole edge base end distance 4D denotes an axial distance between the base end edge of the side hole 3 and the front end of the fixing portion. Specifically, it is preferable that the hole edge base end distance 4D is not less than the hole diameter 3D of the side hole 3. Although the umbrella body 23 and the outer circumference of the catheter main body 1 form a narrow angle area therebetween, the above structure and size are preferable to prevent foreign substances in a blood vessel from being caught there.

Other main structures are substantially equal to those of Embodiment 1.

### Embodiment 6

FIG. 13 is an explanatory diagram of a side view in partial section of the catheter for removing foreign substances in a blood vessel in a release state S1 according to Embodiment 6 of the present invention. The spread ribs 232 of Embodiment 6 intersect with each other to form diamond crossing in the release state S1 so as to make an oblique lattice network formation. Other main structures are equal to those of Embodiment 5.

### Embodiment 7

FIG. 14 is an explanatory diagram of a side view in partial section of the catheter for removing foreign substances in a blood vessel in a release state S1 according to Embodiment 7 of the present invention. The spread ribs 232 of this embodiment include first spread ribs 232a, each of which has a memory of shape of a curved line wherein the front side extends toward the outer circumference; second spread ribs 232b, each of which has a memory of shape of a curved line which extends to reduce the diameter toward the front end from the base end connecting with the front end of the first spread rib 232: a first spread auxiliary rib 232c, which has an annular shape spacing from the catheter main body 1 at the front end of the second spread ribs 232b; and second spread auxiliary ribs 232d, each of which has a memory of shape of an arc curved line on the side of the base end to support the release state S1 of the first spread ribs 232a. The spread sheet 231 of Embodiment 7 covers the first spread ribs 232a among them is fixed to the second spread auxiliary ribs 232d. The spread sheet 231 has a open edge at the boundary between the first spread ribs 232a and the second spread ribs 232b. Other main structures are equal to those of Embodiment 5.

The present invention is not limited to the above-described embodiments and various modifications will become possible without departing from the scope thereof.

What is claimed is:

1. A catheter for removing foreign substances in a blood vessel comprising: a catheter main body; an expansion part that is provided spaced from a front end of the catheter main body at a predetermined front end distance and expands a contour of the catheter; and a side hole for suction of foreign substances in a blood vessel that is provided on a side face of the catheter main body within an area of the front end distance.

2. The catheter for removing foreign substances in a blood vessel according to claim 1, wherein the side hole has a plurality of holes which are provided equally spaced centering an extension axis of the catheter main body.

3. The catheter for removing foreign substance in a blood vessel according to claim 1, wherein the expansion part includes a flow path for fluid or gas that is annularly disposed along an outer circumference of the catheter main body, and an inflating and deflating bag that communicates with a front part of the flow path and is annularly disposed along the outer circumference of the catheter main body, and expandable and contractible in response to inflow and outflow of liquid or gas passing through the flow path.

4. The catheter for removing foreign substance in a blood vessel according to claim 2, wherein the expansion part include a flow path for fluid or gas that is annularly disposed along an outer circumference of the catheter main body, and an inflating and deflating bag that communicates with a front part of the flow path and is annularly disposed along the outer circumference of the catheter main body, and expandable and contractible in response to inflow and outflow of liquid or gas passing through the flow path.

5. The catheter for removing foreign substances in a blood vessel according to claim 1, wherein the expansion part includes an umbrella body with a spread sheet that is disposed on the outer circumference of the catheter main body, and a binder for holding the umbrella body in a binding state in which the umbrella body is being bound along the side face of the outer circumference of the catheter main body, wherein the spread sheet spreads further outwardly from the side face of the outer circumference of the catheter main body so as to block a bloodstream in a release state where the umbrella body is not bound by the binder, and relaxes into a binding state where the umbrella body is bound by the binder.

6. The catheter for removing foreign substances in a blood vessel according to claim 2, wherein the expansion part includes an umbrella body with a spread sheet that is disposed on the outer circumference of the catheter main body, and a binder for holding the umbrella body in a binding state in which the umbrella body is being bound along the side face of the outer circumference of the catheter main body, wherein the spread sheet spreads further outwardly from the side face of the outer circumference of the catheter main body so as to block a bloodstream in a release state where the umbrella body is not bound by the binder, and relaxes into a binding state where the umbrella body is bound by the binder.
